(19) United States
(12) Patent Application Publication
(10) Pub. No.: US 2011/0200566 A1
(43) Pub. Date: Aug. 18, 2011

(54) Compositions and Methods for the Treatment of Vitiligo

(76) Inventors: Xiaowei Xu, Monmounth Junction, NJ (US); Hong Yu, Miami, FL (US)

(21) Appl. No.: 13/121,572

(22) PCT Filed: Sep. 29, 2009
(86) PCT No.: PCT/US09/58729

§ 371 Date: Apr. 29, 2011

Related U.S. Application Data
(60) Provisional application No. 61/101,388, filed on Sep. 30, 2008.

(51) Int. Cl. A61K 35/12; C12N 5/071; A61P 17/00

(52) U.S. Cl. 424/93.7; 435/325

(57) ABSTRACT

Compositions and methods for darkening skin and treating vitiligo are provided.

This application claims priority under 35 U.S.C. §119(e) to U.S. Provisional Patent Application No. 61/101,388, filed on Sep. 30, 2008. The foregoing application is incorporated by reference herein.

### FIELD OF THE INVENTION

The present invention relates to the fields of skin pigmentation and vitiligo therapies. Specifically, methods and compositions for darkening skin and the treatment of vitiligo are provided.

### BACKGROUND OF THE INVENTION

Several publications and patent documents are cited throughout the specification in order to describe the state of the art to which this invention pertains. Each of these citations is incorporated herein by reference as though set forth in full.

Vitiligo is a skin condition resulting from loss of melanocytes in the skin. Autoimmunity has been proposed as a cause for this disease. As a result of the loss of melanocytes, white patches of skin appear on different parts of the body. Any part of the body may be affected, although hair color is usually not affected. In the United States, 2 to 5 million people have the disorder and about 1 to 2 percent of the world's population is affected by this disease. Vitiligo is more common or appears more pronounced on darker skin. It affects people of both sexes equally, and it affects all races. Vitiligo can begin at any age, though about fifty percent of people with vitiligo develop it before the age of twenty five. Vitiligo can cause extreme distress to sufferers because of its unusual appearance.

There are a number of treatment options currently available including include medical, surgical, and other treatments. However, some treatments are not right for everyone and many treatments can have unwanted side effects. Current treatments can also take a long time to work and are not always effective. Most treatments are aimed at restoring color to the white patches of skin. Medical treatment includes steroid creams with or without ultraviolet A (UVA) light and psoralen plus UVA (PUVA). Surgical treatment includes skin grafts from a person's own tissues or autologous melanocytes transfer. More specifically, skin is taken from one area of a patient's body and attached to another area. Alternatively, melanocytes are isolated from the skin and transferred to the affected area. However, the duration of treatment by transplanting melanocytes alone is questionable as they are terminally differentiated cells. The efficacy of all of the above treatments is limited as not every individual responds to these treatments. Notably, during the recovery phase of vitiligo or after successful treatment of vitiligo, the pigmentation often first occurs in the area surround hair follicles, suggesting that follicular melanocytes is critical for vitiligo treatment.

### SUMMARY OF THE INVENTION

In accordance with one aspect of the instant invention, isolated hair follicle derived melanoblasts are provided. Compositions comprising hair follicle derived melanoblasts and at least one pharmaceutically acceptable carrier are also provided. The compositions may further comprise hair follicle derived melanocytes.

Methods of producing hair follicle derived melanoblasts are also provided. In a particular embodiment, the method comprises culturing hair follicle cells in medium void of cholera toxin and 12-O-tetradecanoylphorbol-13-acetate and, optionally, isolating the generated melanoblasts. In another embodiment, the culture medium comprises endothelin-3, stem cell factor, and basic fibroblast growth factor.

In accordance with another aspect of the instant invention, methods for treating vitiligo are provided. The methods comprise administering to a patient in need thereof melanoblasts, particularly hair follicle derived melanoblasts.

### BRIEF DESCRIPTIONS OF THE DRAWING

FIG. 1A is an image of melanoblasts (arrow) and melanocytes (arrow head) after culturing hair follicle derived cells in the melanoblast medium for 2 weeks. FIG. 1B is an image of Fontana-Mason staining showing melanin pigment in these cultured cells.

FIGS. 2A-2C provide images demonstrating that hair derived melanocytes and melanoblasts are functional as epidermal melanocytes. FIG. 2A is Fontana-Mason stain showing that pigment producing melanocytes are present in the dermal epidermal junction (arrow points to a melanocyte). FIG. 2B is an image of keratinocytes which also contain melanin pigment, thereby indicating that melanocytes can transfer pigment to keratinocytes (arrow points to melanin pigment in keratinocytes). FIG. 2C demonstrates that the hair derived cells also express tyrosinase (arrow points to tyrosinase positive cells).

### DETAILED DESCRIPTION OF THE INVENTION

Replenishing melanocytes selectively in vitiliginous macules by autologous melanocytes from skin has been tried with some success. Vitiligo has also been treated by autologous, noncultured melanocyte-keratinocyte cell transplantation. Surgical treatment for vitiligo through grafting of entire hair follicles has also been tried. However, as stated hereinabove, autoimmunity has been proposed as a cause for this disease. Melanoblasts (melanocyte precursors) are more resistant to immuno-destruction. As such, the transplant of both melanocytes and melanoblasts from hair follicles to areas with vitiligo has a better effect than transplantation of melanocytes only. Similar to normal epidermal melanocytes, human hair follicle derived melanocytes and melanoblasts migrate to the dermal-epidermal junction (see Example). The introduced melanocytes express melanocyte lineage markers and are able to transfer melanin to keratinocytes, thereby indicating their ability to darken the white patches of skin associated with vitiligo.

In accordance with one aspect of the instant invention, methods for darkening skin are provided, such as for the treatment of vitiligo or other skin disorders/conditions lacking melanin and/or needing darkening. The methods comprise administering a composition comprising melanoblasts and at least one pharmaceutically acceptable carrier to a patient in need thereof. In a preferred embodiment, the melanoblasts are obtained from the patient to be treated. In a particular embodiment, the composition also comprises melanocytes. In a preferred embodiment, the melanocytes and melanoblasts are isolated/obtained from human hair follicles. In a particular embodiment, the composition also comprises keratinocytes (e.g., autologous keratinocytes). Compositions of the instant invention may be delivered to the areas of vitiligo by any means (e.g., injections, transplants). The compositions may be delivered to the dermal-epidermal junction. For example, the compositions of the instant invention may be delivered to artificially induced blisters (e.g., suction blisters) or delivered via artificially generated pores/voids (see e.g., U.S. Patent Application Publication No. 2007/0225779). The treated area can then be observed for repigmentation. The melanoblasts, being more resistant to the autoimmune reaction, give long term repigmentation. Melanoblasts (e.g., compositions of the instant invention) can be re-administered, if needed. In a particular embodiment, the melanoblasts are administered until a desired level of pigmentation and color is obtained (i.e., the melanoblasts are administered until the treated area matches the normal existing skin).

In accordance with another aspect of the instant invention, methods of obtaining hair follicle derived melanocytes and melanoblasts are provided. Hair follicles may be obtained from the patient to be treated (e.g., by punch biopsy). Preferably, the melanocytes and melanoblasts are isolated in a medium devoid of cholera toxin and 12-O-tetradecanoylphorbol-13-acetate (TPA). In a particular embodiment, a medium comprising endothelin-3 (EDN-3), stem cell factor (SCF), and basic fibroblast growth factor (bFGF) can be used to isolate the melanocytes and melanoblasts. The hair derived melanocytes and melanoblasts can then, optionally, be grown and expanded.

As stated hereinabove, melanoblasts are melanocyte precursors. As demonstrated hereinbelow, melanoblasts and melanocytes are readily identifiable by microscopy (see Example). Examples of melanoblast markers include, without limitation, tyrosinase-related protein-2/DOPAchrome tautomerase (TRP-2/DT) and BRN2 (also known as POU3F2 and N-Oct-3) (Steel et al. (1992) Development, 115:1111-1119; Cook et al. (2003) J. Invest. Derm., 121:1150-1159). Examples of melanocyte markers include, without limitation, MITF (microphthalmia-associated transcription factor), gp100, tyrosinase, and Melan-A (MART-1).

In accordance with another embodiment of the instant invention, at least one other method for the treatment of vitiligo (e.g., administering a steroid creams with or without ultraviolet A (UVA) light; administering psoralen with UVA (PUVA); skin grafts; transplanting melanocytes alone) may be used with instant methods for darkening skin and treating vitiligo. Other examples of methods for the treatment of vitiligo are provided in U.S. Patent Application Publication Nos. 2001/0044422, 2002/0013609, 2004/0061142, 2005/0148017, 2007/0027080, and 2008/0207733. The vitiligo treatment method of the instant invention may be performed before, after, or concurrently with the other vitiligo treatment methods.

### Definitions

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, matrix, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Carriers (e.g., matrices) may influence the physical state, stability, rate of in vivo release, and rate of in vivo clearance of components of a pharmaceutical composition of the present invention. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

A “therapeutically effective amount” of a compound or a pharmaceutical composition refers to an amount effective to prevent, inhibit, treat, or lessen the symptoms of a particular disorder or disease.

The term “treat” as used herein refers to any type of treatment that imparts a benefit to a patient afflicted with a disease, disorder, or condition, including improvement in the condition of the patient (e.g., in one or more symptoms), delay in the progression of the condition, etc. With regard to vitiligo, the term “treat” includes darkening the white/lighter patches of skin associated with the disease.

The term “culturing” refers to growing or maintaining a population of cells under suitable conditions in a medium.

The term “isolated” refers to a cell or group of cells which are not associated with one or more cells or one or more cellular components that are associated with the cell or group of cells in vivo.

The following example provides illustrative methods of practicing the instant invention, and is not intended to limit the scope of the invention in any way.

### EXAMPLE

Human scalp tissues (1×2 cm² or less) were obtained through Cooperative Human Tissue Network with approval from the Internal Review Boards of the University of Pennsylvania. Tissues were rinsed, trimmed to remove connective tissues, cut into small pieces and subjected to enzymatic dissociation in 4.8 mg/ml dispase in DMEM for 24 hours at 4° C. After treatment, the epidermis was peeled off from the dermis, and hairs were then plucked from the dermis. Hairs were rinsed thoroughly with PBS and examined under a microscope to prevent contaminating epidermal or dermal cells.

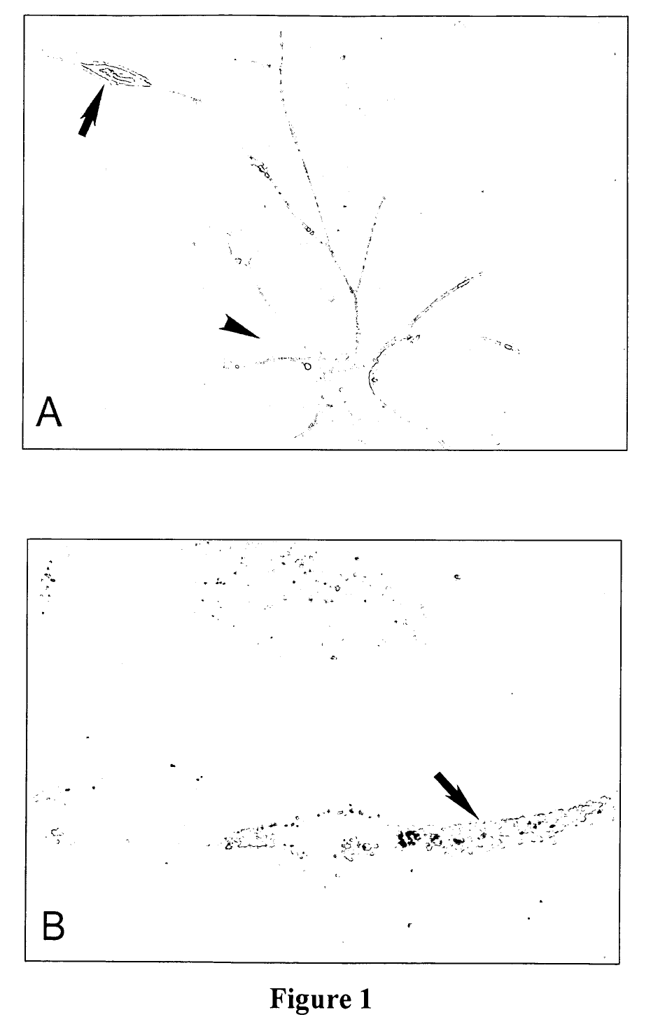

To obtain single cells from follicular epithelium, hairs were treated twice with 0.25% trypsin/EDTA (Invitrogen) for 30 minutes at 37° C. The cell suspension was filtered through a 40-μm cell strainer (BD Biosciences, Franklin Lake, N.J.) and counted. Single cells were cultured in melanoblast medium (per 512 ml, MCDB 153 medium 80%, fetal bovine serum 20%, chelated FBS 2%, L-glutamine 5 ug/ml, cholera toxin 15 ug/ml, basic fibroblast growth factor (bFGF) 0.5 ng/ml, endothelin-3 (ET3) 100 nM, stem cell factor (SCF) 10 ng/ml). Melanoblasts and melanocytes can be seen in the culture after 2 weeks (FIG. 1A). The cultured cells contain melanin pigment similar to epidermal melanocytes (FIG. 1B).

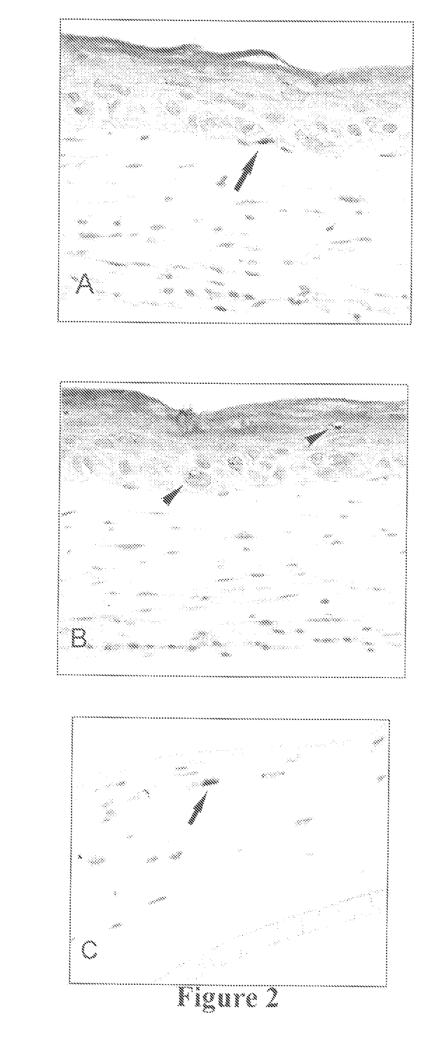

The hair follicle derived melanocytes and melanoblasts were introduced into a human skin reconstruct that mimics human skin architecture. This model has been used extensively to study interactions among melanocytes, epidermal keratinocytes and dermal fibroblasts. Similar to normal epidermal melanocytes, the hair follicle derived melanocytes and melanoblasts migrated to the dermal-epidermal junction (FIG. 2A). These melanocytes can transfer melanin pigment to keratinocytes (FIG. 2B), and these melanocytes express melanocyte lineage markers, such as tyrosinase (FIG. 2C). These data indicate that hair derived melanocytes and melanoblasts have normal function as epidermal melanocytes.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated hair follicle derived melanoblast.

2. A composition comprising the hair follicle derived melanoblasts of claim 1 and at least one pharmaceutically acceptable carrier.

3. The composition of claim 2 further comprising hair follicle derived melanocytes.

4. A method of treating vitiligo, said method comprising administering to a patient in need thereof the composition of claim 2.

5. A method of darkening skin, said method comprising administering to a patient in need thereof the composition of claim 2.

6. A method of producing the hair follicle derived melanoblast of claim 1, said method comprising:
a) obtaining hair follicles from a patient;
b) culturing the cells of the hair follicle obtained in step a) in medium void of cholera toxin and 12-O-tetradecanoylphorbol-13-acetate; and
c) optionally isolating the generated melanoblasts.

7. The method of claim 6, wherein the medium of step b) comprises endothelin-3, stem cell factor, and basic fibroblast growth factor.
